(19) United States
(12) Patent Application Publication
(10) Pub. No.: US 2004/0176370 A1
(43) Pub. Date: Sep. 9, 2004

(54) 3,4-Dihdro-N-[[1-(3-hydroxybutyl)-4-piperidinyl]methyl]-2H-[1,3]oxazino[3,2-a]indole-10-carboxamide as 5-HT(4) receptor antagonist

(75) Inventors: Frank Hossner, Harlow (GB); David Austin Ryan, Hitchin (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/801,510

(22) Filed: Mar. 16, 2004

Related U.S. Application Data
(63) Continuation of application No. 09/787,980, filed on Apr. 18, 2002, now abandoned.
Application No. 09/787,980 is a 371 of international application No. PCT/EP99/06780, filed on Sep. 14, 1999.

(30) Foreign Application Priority Data
Sep. 17, 1998 (GB) 9820294.8

(51) Int. Cl. A61K 31/54; A61K 31/535; C07D 491/04

(52) U.S. Cl. 514/230.2; 544/89

(57) ABSTRACT

3,4-Dihydro-N-[[1-(3-hydroxybutyl-4-piperidinyl]methyl]-2H-[1,3]oxazino[3,2-α]indole-10-carboxamide and its use as 5-HT₄ receptor antagonist for example in the treatment or prophylaxis of gastrointestinal disorders, cardiovascular disorders and CNS disorders.

This invention relates to novel compounds having pharmacological activity, to a process for their preparation and to their use as pharmaceuticals.

EP-A429984 (Nisshin Flour Milling Co., Ltd.) describes indole derivatives having 5-HT₃ receptor antagonist activity.

European Journal of Pharmacology 146 (1988), 187-188, and Naunyn-Schmiedeberg's Arch. Pharmacol. (1989) 340:403-410, describe a non classical 5-hydroxytryptamine receptor, now designated the 5-HT₄ receptor, and that ICS 205-930, which is also a 5-HT₃ receptor antagonist, acts as an antagonist at this receptor.

WO 91/16045 (SmithKline and French Laboratories Limited) describes the use of cardiac 5-HT₄ receptor antagonists in the treatment of atrial arrhythmias and stroke.

EP-A-501322 (Glaxo Group Limited) describes indole derivatives having 5-HT₄ antagonist activity.

WO93/18036 (SmithKline Beecham plc) describes certain condensed indole derivatives having 5-HT₄ receptor antagonist activity. Among these derivatives the compound N-[(1-ⁿbutyl-4-piperidyl)methyl]-3,4-dihydro-2H-[1,3]oxazino[3,2-a]indole-10-carboxamide has been found to have potent 5-HT₄ receptor antagonist activity and to be of therapeutic value in the treatment of irritable bowel syndrome. In the evaluation of this compound we have found that one of its metabolites, namely the compound of formula (I) below, itself has potent 5-HT₄ receptor antagonist activity.

Accordingly, the present invention provides 3,4dihydro-N-[[1-(3-hydroxybutyl)-4-piperidinyl]methyl]-2H-[1,3]oxazino[3,2-α]indole-10-carboxamide i.e. the compound of formula (I), or a pharmaceutically acceptable salt thereof:

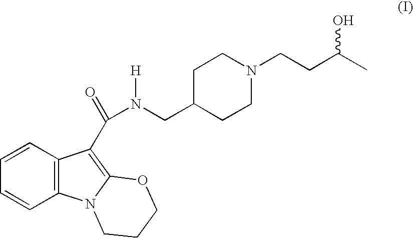

According to a further feature of the present invention we provide the compound of formula (I) and its pharmaceutically acceptable salts in synthetic form, i.e. not produced by a biological process from its metabolic precursor referred to above. The compound of formula (I) is also preferably in pharmaceutically acceptable form. By pharmaceutically acceptable form is meant, inter alia, of a pharmaceutically acceptable level of purity excluding normal pharmaceutical additives such as diluents and carriers, and including no material considered toxic at normal dosage levels. A pharmaceutically acceptable level of purity will generally be at least 50% excluding normal pharmaceutical additives, preferably 75%, more preferably 90% and still more preferably 95%. One preferred pharmaceutically acceptable form is the crystalline form, including such form in a pharmaceutical composition.

The pharmaceutically acceptable salts of the compound of the formula (I) include acid addition salts with conventional acids such as hydrochloric, hydrobromic, boric, phosphoric, sulphuric acids and pharmaceutically acceptable organic acids such as acetic, tartaric, maleic, citric, succinic, benzoic, ascorbic, methanesulphonic, α-keto glutaric, α-glycerophosphoric, and glucose-1-phosphoric acids.

Examples of pharmaceutically acceptable salts include quaternary derivatives of the compound of formula (I) such as the compounds quaternised by compounds Rₓ-T wherein Rₓ is C₁₋₆ alkyl, phenyl-C₁₋₆ alkyl or C₅₋₇ cycloalkyl, and T is a radical corresponding to an anion of an acid. Suitable examples of Rₓ include methyl, ethyl and n- and iso-propyl; and benzyl and phenethyl. Suitable examples of T include halide such as chloride, bromide and iodide.

Examples of pharmaceutically acceptable salts also include internal salts such as N-oxides.

The compound of the formula (I) and its pharmaceutically acceptable salts, (including quaternary derivatives and N-oxides) may also form pharmaceutically acceptable solvates, such as hydrates, which are included wherever the compound of formula (I) or a salt thereof is herein referred to. The compound of formula (I) is a racemate. The present invention also covers the corresponding individual (+) and (−) enantiomers.

The compound of formula (I) may be prepared as described in the Example below or by other conventional coupling of the indole moiety with the piperidine side chain for example by reacting 1-(3-(protected)hydroxybutyl)-4-piperidinyl methylamine with methyl 3,4-dihydro-2H-[1,3]oxazino[3,2-a]indole-10-carboxylate and subsequently removing the protecting group: the 3-hydroxybutyl group is advantageously protected by an appropriate silyl or benzyl group. Suitable methods are as described in GB 2125398A (Sandoz Limited), GB 1593146A and EP-A-36269 (Beecham Group p.l.c.), EP-A429984 (Nisshin Flour Milling Co.) and EP-A-328200 (Merck Sharp & Dohme Limited). Reference is also made to EP-A-501322 (Glaxo Group Limited). It will be appreciated that the formation of O-containing ring may be carried out before or after coupling.

The compounds of the present invention are 5-HT₄ receptor antagonists and it is thus believed may generally be used in the treatment or prophylaxis of gastrointestinal disorders, cardiovascular disorders and CNS disorders.

They are of potential interest in the treatment of irritable bowel syndrome (IBS), in particular the diarrhoea aspects of IBS, i.e., these compounds block the ability of 5-HT to stimulate gut motility via activation of enteric neurones. In animal models of IBS, this can be conveniently measured as a reduction of the rate of defaecation. They are also of potential use in the treatment of urinary incontinence which is often associated with IBS.

They may also be of potential use in other gastrointestinal disorders, such as those associated with upper gut motility, and as antiemetics. In particular, they are of potential use in the treatment of the nausea and gastric symptoms of gastro-oesophageal reflux disease and dyspepsia Antiemetic activity is determined in known animal models of cytotoxic-agent/radiation induced emesis.

Specific cardiac 5-HT₄ receptor antagonists which prevent atrial fibrillation and other atrial arrhythmias associated with 5-HT, would also be expected to reduce occurrence of stroke (see A. J. Kaumann 1990, Naumyn-Schrmiedeberg's Arch. Pharmacol. 342, 619-622, for appropriate animal test method).

It is believed that platelet-derived 5-HT induces atrial arrhythmias which encourage atrial fibrillation and atrial disorders are associated with symptomatic cerebral and sytemic embolism. Cerebral embolism is the most common cause of ischaemic stroke and the heart the most common source of embolic material. Of particular concern is the frequency of embolism associated with atrial fibrillation.

Anxiolytic activity is likely to be effected via the hippocampus (Dumuis et al 1988, Mol Pharmacol., 34, 880-887). Activity may be demonstrated in standard animal models, the social interaction test and the X-maze test.

Migraine sufferers often undergo situations of anxiety and emotional stress that precede the appearance of headache (Sachs, 1985, Migraine, Pan Books, London). It has also been observed that during and within 48 hours of a migraine attack, cyclic AMP levels are considerably increased in the cerebrospinal fluid (Welch et al., 1976, Headache 16, 160-167). It is believed that a migraine, including the prodomal phase and the associated increased levels of cyclic AMP are related to stimulation of 5-HT₄ receptors, and hence that administration of a 5-HT₄ antagonist is of potential benefit in relieving a migraine attack.

The invention also provides a pharmaceutical composition comprising the compound of formula (I), or a pharmaceutically acceptable salt thereof, and a pharmaceutically acceptable carrier.

Such compositions are prepared by admixture and are usually adapted for enteral such as oral, nasal or rectal, or parenteral administration, and as such may be in the form of tablets, capsules, oral liquid preparations, powders, granules, lozenges, reconstitutable powders, nasal sprays, suppositories, injectable and infusable solutions or suspensions. Sublingual or transdermal administration is also envisaged. Orally administrable compositions are preferred, since they are more convenient for general use.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well known methods in the art, for example with an enteric coating.

Suitable fillers for use include cellulose, mannitol, lactose and other similar agents. Suitable disintegrants include starch, polyvinylpolypyrrolidone and starch derivatives such as sodium starch glycollate. Suitable lubricants include, for example, magnesium stearate.

Suitable pharmaceutically acceptable wetting agents include sodium lauryl sulphate. Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents.

Oral liquid preparations are usually in the form of aqueous or oily suspensions, solutions, emulsions, syrups, or elixirs or are presented as a dry product for reconstitution with water or other suitable vehicle before use. Such liquid preparations may contain conventional additives such as suspending agents, emulsifying agents, non-aqueous vehicles (which may include edible oils), preservatives, and flavouring or colouring agents.

The oral compositions may be prepared by conventional methods of blending, filling or tabletting. Repeated blending operations may be used to distribute the active agent throughout those compositions employing large quantities of fillers. Such operations are, of course, conventional in the art.

For parenteral administration, fluid unit dose forms are prepared containing a compound of the present invention and a sterile vehicle. The compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum.

Parenteral suspensions are prepared in substantially the same manner except that the compound is suspended in the vehicle instead of being dissolved and sterilised by exposure of ethylene oxide before suspending in the sterile vehicle. Advantageously, a surfactant or wetting agent is included in the composition to facilitate uniform distribution of the compound of the invention.

The invention further provides a method of treatment of irritable bowel syndrome, gastro-oesophagal reflux disease, dyspepsia, atrial arrhythmias and stroke, anxiety and/or migraine in mammals, such as humans, which comprises the administration of an effective amount of the compound of the formula (I) or a pharmaceutically acceptable salt thereof. In particular, the method comprises treatment of IBS or atrial arrhythmias and stroke.

An amount effective to treat the disorders hereinbefore described depends on the relative efficacies of the compounds of the invention, the nature and severity of the disorder being treated and the weight of the mammal. However, a unit dose for a 70 kg adult will normally contain 0.05 to 1000 mg for example 0.5 to 500 mg, of the compound of the invention. Unit doses may be administered once or more than once a day, for example, 2, 3 or 4 times a day, more usually 1 to 3 times a day, that is in the range of approximately 0.0001 to 50 mg/kg/day, more usually 0.0002 to 25 mg/kg/day.

No adverse toxicological effects are indicated within the aforementioned dosage ranges.

The invention also provides the compound of formula (I) or a pharmaceutically acceptable salt thereof for use as an active therapeutic substance, in particular for use as a 5-HT₄ receptor antagonist in the treatment of the disorders hereinbefore described.

The invention also provides the use of the compound of formula (I) in the manufacture of a medicament for use as a 5-HT₄ receptor antagonist in the treatment of the disorders hereinbefore described.

The following Example illustrates the preparation of the compound of formula (I) by two methods, as indicated in the scheme below:

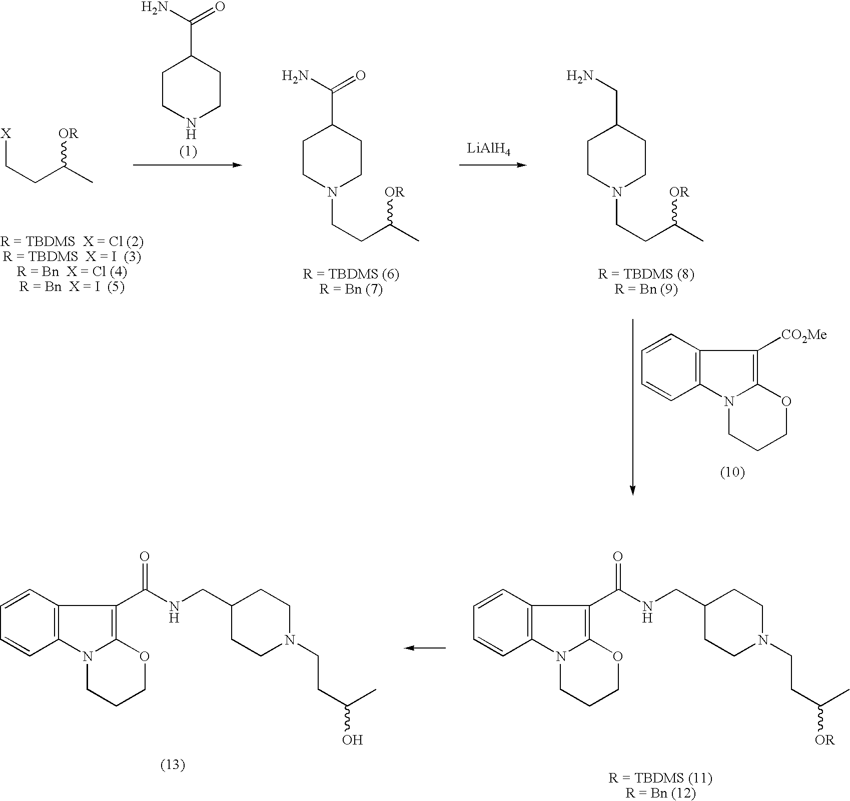

(1) 4-piperidinecarboxamide

(2) (3-chloro-1 -methylpropoxy)(1,1-dimethylethyl)dimethylsilane

(3) (1,1-dimethylethyl)(3-iodo-1-methylpropoxy)dimethylsilane

(4) 1-[(3-chloro-1-methylpropoxy)methyl]benzene

(5) 1-[(3-iodo-1-methylpropoxy)methyl]benzene

(6) 1-[3-[[(1,1-dimethylethyl)dimethylsiyl]oxy]butyl]-4-piperidinecarboxamide

(7) 1-[3-(phenylmethoxy)butyl]-4-piperidinecarboxamide

(8) 1-[3-[[(1,1-dimethyethyl)dimethylsilyl]oxy]butyl]-4-piperidinemethanamine

(9) 1-[3-(phenylmethoxy)butyl]-4-piperidinemethanamine

(10) methyl 3,4-dihydro-2H-[1,3]oxazino[3,2-α]indole-10-carboxamide

(11) N-[[1-[3-[[(1,1-dimethylethyl)dimethylsilyl]oxy]butyl]-4-piperidinyl]methyl]-3,4-dihydro-2H-[1,3]oxazino[3,2-α]indole-10-carboxamide

(12) 3,4-dihydro-N-[[1-[3-(phenylmethoxy)butyl] -4-piperidinyl]methyl]-2H-[1,3]oxazino[3,2-α]indole-10-carboxamide

(13) 3,4-dihydro-N-[[1-(3-hydroxybutyl)-4-piperidinyl]methyl]-2H-[1,3]oxazino[3,2-α]indole-10-carboxamide

### Method 1

Preparation of (2)

To 4-chlorobutan-2-ol (15 g, 0.14M) dissolved in DMF (50 ml) was added imidazole (20.7 g, 0.3M), and TBDMS chloride (22.9 g, 0. 15M). The solution formed was stirred under nitrogen for 16 hr., quenched with water (200 ml) and extracted with ethyl acetate (2×100 ml). The combined organic extracts were washed with water (5×50 ml), brine, dried over sodium sulphate, filtered and evaporated under reduced pressure to give an oil. Purification by Kugelrohr distillation gave (2) as an oil, 27.4 g (89%).

¹H NMR (CDCl₃) δ: 3.92 ((m, 1H), 3.32 (m, 2H), 1.76 (m, 2H), 1.08 (d, 3H), 0.81 (s, 9H), 0.00 (s, 6H).

Preparation of (3)

A solution of (2) (10 g, 44.9 mmol) and sodium iodide (16.6 g, 111 mmol) in acetone (50 ml) was heated under reflux for 45 hr. The mixture was allowed to cool, filtered, and the filtrate evaporated under reduced pressure. The residue was extracted with diethyl ether (5×50 ml) and the combined extracts evaporated under reduce pressure to give (3) as an oil, 13.5 g (96%).

¹H NMR (CDCl₃) δ: 3.80 (m, 1H), 3.12 (m, 2H), 1.82 (m, 2H), 1.08 (d, 3H), 0.77 (s, 9H), 0.01 (d, 6H).

Preparation of (6)

A suspension of iso-nipecotamide (1) (3 g, 23.4 mmol), (3) (8.2 g, 26.2 mmol) and anhydrous potassium carbonate (9.7 g, 70 mmol) in toluene (120 ml) was heated at 90° C. for 17 hours, then heated under reflux for 5 hr. The reaction mixture was allowed to cool, filtered, and the filtrate washed with water (4×30 ml), brine, dried over sodium sulphate, filtered, and evaporated under reduced pressure to an oil which solidified on standing, obtaining (6), 7.9 g (108%). This material was used without further purification.

¹H NMR (CDCl₃) δ: 5.8-5.6 (d, broad, 2H), 3.8 (m, 1H), 2.9 (m, 2H), 2.4-1.5 (m, 11H), 1.1 (d, 3H), 0.82 (s, 9H), 0.01 (s, 6H).

¹³C NMR (CDCl₃) δ: 181, 68, 56, 54, 43, 37, 29, 26, 24, 19, −4.2, −4.6.

m.s. 315.2 (ES⁺, M+1).

Preparation of (8)

To a suspension of LiAlH₄ (11.6 g, 0.24M) in THF (115 ml) at 5° C. was added (6) (7 g, 0.022M) dissolved in THF (65 ml) dropwise over 1 hour. The mixture was allowed to warm up to room temperature, stirred for 30 minutes and then heated at reflux for 1 hour. The reaction mixture was cooled to ambient temperature, then sodium hydroxide solution (7.9M, 22.5 ml) was added dropwise over 2 hours. The resulting mixture was filtered through celite, the celite washed with THF (4×50 ml), and the combined filtrate and washes evaporated under reduced pressure to give (8) as an oil, 3.1 g (46%).

¹H NMR (CDCl₃) δ: 3.8 (m, 1H), 3.1-1.1 (m, 20H), 0.8 (s, 9H), 0.0 (s, 6H).

m.s. 301.3 (ES⁺, M+1).

Preparation of (11)

Trimethylaluminium (2M in toluene, 8.2 ml) was diluted with toluene (8 ml) and the solution cooled to 0° C. A solution of (8) (3 g, 10 mmol) in toluene (25 ml) was added over 3 minutes, followed by SB-224908 (3.4 g, 14.7 mmol). The mixture was heated at reflux for 5 hours, then allowed to cool, and sodium hydroxide solution (10% w/w, 18 ml) was added dropwise. The toluene layer was washed with water, brine, dried over sodium suphate, filtered, and evaporated under reduced pressure to give an oil. This was purified by flash chromatography on silica (eluting with 0-1% MeOH in CHCl₃) obtaining 2 g (40%) (11) as a solid.

¹H NMR (CDCl₃, 400 MHz) δ: 8.3 (m 1H), 7.2-7.1 (m, 3H), 6.5 (t, 1H, NH), 4.5 (t, 2H), 4.1 (t, 2H), 3.8 (m, 1H), 3.3 (t, 2H), 2.9 (m, 2H), 2.3 (m, 4H), 1.9 (m, 2H), 1.7 (m, 2H), 1.6 (m, 2H), 1.4 (m, 2H), 1.1 (d, 3H), 0.9 (s, 9H), 0.04 (s, 6H).

¹³C (CDCl₃) δ: 165, 149, 131, 125, 122, 121, 120, 107, 89, 67, 66, 65, 55, 53.8, 53.6, 44, 39, 36.8, 36.5, 30, 25, 24, 21, 18, 0.0, −4.3, −4.7.

m.s. 500.2 (AP⁺, M+1).

Preparation of (13)

A solution of (11) (0.2 g, 0.4 mmol) is acetic acid/water (2:1, 20.8 ml) was heated at 75° C. for 5½ hr under nitrogen. The reaction mixture was evaporated under reduced pressure to give an oil which was redissolved in chloroform (30 ml) and washed with sodium bicarbonate solution. The chloroform layer was dried over sodium sulphate, filtered, and the filtrate evaporated to a foam (0.16 g). This was crystallised from IPA/Et₂O obtaining (13), 0.057 g (37%), m.pt. 126-7° C.

¹H NMR (CDCl₃, 400 MHz) δ: 8.3 (m, 1H), 7.26-7.08 (m, 3H), 6.5 (t, 1H, NH), 4.5 (t, 2H), 4.1 (t, 2H), 3.9 (m, 1H), 3.3 (m, 2H), 3.2 (m, 1H), 2.9 (m, 1H), 2.6-2.5 (m, 2H), 2.4 (m, 2H), 2.1 (m, 1H), 1.8-1.3 (m, 10H), 1.26 (d, 3H).

¹³C NMR(CDCl₃) δ: 165, 149, 131, 126, 122, 121, 120.7, 108, 89, 70, 67, 58, 55, 52, 44, 39, 36, 33, 30.4, 29.9, 23, 21.

m.s. (3.86.1, M+1).

R_{f} 0.5 (SiO₂, 5:1:1 EtOAc: AcOH: NH₄OH).

### Method 2

Preparation of (4)

To a mixture of benzyl-2,2,2-trichloroacetimidate (50.6 g, 0.2M) and 4-chloro-butan-2-ol (19.8 g, 0.18M) in cyclohexane/dichloromethane (2:1, 106.5 ml) at 5° C. was added triflic acid (1.2 ml, 0.014M) dropwise with stirring under nitrogen over 30 minutes. There was an immediate precipitate and an exotherm to 30° C. The mixture was cooled to 5° C. and stirred for 5 hours, then allowed to warm to room temperature, filtered, the filtrate washed with sodium bicarbonate solution, brine, dried over soium sulphate, filtered and the filtrate evaporated under reduced pressure to give (4) as an oil, 46.4 g (128%). This material was used without further purification.

¹H NMR (CDCl₃) δ: 7.4-7.2 (m, 5H+CHCl₃), 4.5 (AB quartet, 2H), 3.7 (m, 3H), 2.0 (m, 2H), 1.2 (d, 3H).

Impurities observed at δ 4.55 (s, 0.23H), 4.0 (m, 0.08H), 1.4 (s, 2.76H, ≡0.23M cyclohexane).

Preparation of (5)

(4) (46.4 g) obtained above was added to sodium iodide (68.3 g, 0.46M) in acetone (250 ml) and the mixture heated under nitrogen at reflux for 25 hours. Further sodium iodide (14 g, 0.09M) dissolved in acetone (50 ml) was added, and the mixture heated under reflux for 16 hours. The reaction mixture was allowed to cool, filtered, the filtrate evaporated under reduced pressure and the residue extracted with hexane (4×50 ml). The combined extracts were washed with 1% aqueous sodium thiosulphate solution (2×50 ml), water, dried over sodium sulphate, filtered, and evaporated under reduced pressure to give (5) as an oil, 51.9 g (98%).

¹H NMR (CDCl₃) δ: 7.3 (m, 5H+CHCl₃), 4.5 (AB quartet, 2H), 3.62 (m, 1H), 3.25 (m, 2H), 2.0 (mn, 2H), 1.25 (d, 3H).

Preparation of (7)

A suspension of iso-nipecotamide (18 g, 0.14M), anhydrous potassium carbonate (58.2 g, 0.42M) and (5) (44.7 g, 0.15M) was heated under nitrogen at reflux for 7 hours. The resulting mixture was subjected to hot filtration and the filtrate evaporated under reduced pressure to give a solid. This was redissolved in ethyl acetate, washed with water and brine, and concentrated until crystallization occurred. The solid was collected by filtration and washed with ethyl acetate and diethyl ether to give (7) 26.1 g (64%).

¹H NMR (CDCl₃) δ: 7.4-7.2 (m, 5H+CHCl₃), 5.5 (s, broad, 2H, NH₂), 4.5 (AB quartet, 2H), 3.6 (m, 1H), 2.9 (m, 2H), 2.4 (m, 2H), 2.15 (m, 1H), 2.0-1.6 (m, 8H), 1.2 (d, 3H).

m.s. 291.2 (ES⁺, M+1).

Preparation of (9)

To a suspension of LiAlH₄ (16.82 g, 0.44M) in THF (250 ml) at 5° C. was added amide (7) (10.34 g, 0.036M) dissolved in THF (150 ml) dropwise over 1 hour. The mixture was allowed to warm to ambient temperature, then stirred for 18 hours under nitrogen. The mixture was cooled to 50° C., aqueous sodium hydroxide solution (7.8M, 33 ml) added dropwise over 30 min, and the mixture filtered through celite. The celite was washed with THF (3×50 ml) and the combined filtrate and washings evaporated under reduced pressure to give (9) as an oil, 9.4 g (96%).

¹H NMR (CDCl₃) δ: 7.4-7.2 (m, 5H+CHCl₃), 4.5 (AB quartet, 2H), 3.6 (m, 1H), 2.9 (m, 2H), 2.6 (d, 2H), 2.4 (m, 2H), 2.0-1.2 (m, 14H).

m.s. 277.2 (ES⁺, M+1).

Preparation of (12)

Trimethylaluminium (2M in toluene, 18 ml) was diluted with toluene (18 ml) and the solution cooled to 0° C. The amine (9) (9.2 g, 33 mmol) dissolved in toluene (30 ml) was added over 3 min, followed by SB-224908 (10) (7.6 g, 33 mmol). The mixture was heated at reflux for 5 hours, then allowed to cool, and sodium hydroxide solution (10% w/w, 80 ml) added dropwise. The toluene layer was washed with water, brine and evaporated under reduced pressure to give an oil (14.7 g). This was purified by flash chromatography on silica (eluting with 0-20% MeOH/CH₂Cl₂) obtaining (12) 9.3 g (60%) as an oil.

¹H NMR (CDCl₃) δ: 8.3 (m, 1H), 7.4-7.1 (m, 8H+CHCl₃), 6.5 (t, 1H, NH), 4.5 (m, 4H), 4.1 (t, 2H), 3.6 (m, 1H), 3.3 (t, 2H), 3.0 (m, 2H), 2.5-2.3 (m, 4H), 2.1-1.6 (m, 7H), 1.5-1.2 (m, 5H).

R_{f} 0.7 (SiO₂, 5:1:1 EtOAc: MeOH: NH₄OH).

Preparation of (13)

(12) (8.2 g, 17 mmol) was dissolved in ethanol (150 ml) and glacial acetic acid (8 ml). Oxalic acid dihydrate (2.4 g, 19 mmol) was added and the mixture hydrogenated over 10% Pd/C (4.8 g) at 45° C. 50 psi for 30 hours. The mixture was allowed to cool, filtered through celite, and the filtrate evaporated under reduced pressure. The residue was partitioned between chloroform (150 ml) and concentrated aqueous potassium carbonate solution (100 mnl). The chloroform layer was washed with water (3×25 ml), brine (25 ml), dried over sodium sulphate, filtered, and the filtrate evaporated under reduced pressure to give an oil (4.7 g). This was crystallized from iso-propanol/diethyl ether obtaining (13) 3 g (40%). The product prepared by this method had identical properties to that obtained using Method 1.

5-HT₄ RECEPTOR ANTAGONIST ACTIVITY

Male Dunkin Hartley guinea-pigs, weighing 200-300 g are used. Longitudinal muscle-myenteric plexus (LMMP) preparations, 2-3 cm long, are obtained from the distal colon region. These are suspended under a 0.5 g load in isolated tissue baths containing Krebs Henseleit solution (NaCl 118 mM, KCl 4.7 mM KH2PO4 1.2 mM MgSO4 7H2O 1.2 mM, Glucose 11.1 mM, NaHCO3 25 mM, CaCl2 6H2O 2.5 mM) bubbled with 5% CO₂ in O₂, maintained at 37° C. and containing 1 uM Granisetron and 0.1 uM Methiothepin to block 5HT₃ and 5HT₁-like receptors respectively. 100 uM Pargyline is also added to the tissues at the start of the experiment. Tissues are left for 15 minutes to equilibrate and then exposed to 5-HT at 0.1 uM every 15 minutes until a uniform response is achieved. Following a half hour period for tissues to stabilise, non-cumulative dose response curves to 5-HT are constructed in all tissues. When base-lines have returned to normal, test compounds are added in the reservoirs of the tissue set-ups and washed in to the tissues. Tissues are incubated with the antagonists for 45 minutes after which a second non-cumulative dose response curve to 5-HT is constructed.

To test for selectivity of action, compounds are tested for their ability to antagonise cholinergically-mediated contractions of the guinea-pic colon, evoked by the nicotinic receptor agonist, DMPP (1,1-dimethyl-4-phenyl-piperazinium iodide). For these experiments, tissues and equipment are set up as for the above, 5HT₄ receptor experiments. After sensitization, non-cumulative dose response curves are constructed to DMPP. Test compounds are incubated with the tissues as above, and a second dose response curve to DMPP created. Results are given as mean pKB±SEM values for each antagonists.

The compound of formula (I) was found to have a pKB value of 9.3 and did not significantly affect DMPP-evoked contractions.

1. 3,4-dihydro-N-[[1-(3-hydroxybutyl)-4-piperidinyl]methyl]-2H-[1,3]oxazino[3,2-α]indole-10-carboxamide i.e. the compound of formula (I), or a pharmaceutically acceptable salt thereof:

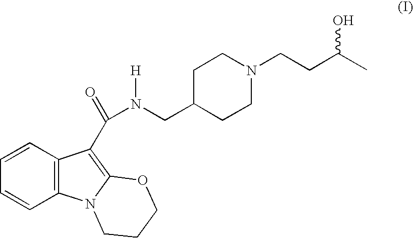

2. The compound of formula (I) and its pharmaceutically acceptable salts as claimed in claim 1 in pharmaceutically acceptable form.

3. The compound of formula (I) and its pharmaceutically acceptable salts as claimed in claim 1 or claim 2 in synthetic form.

4. A process for preparing a compound of formula (I) according to claim 1 which comprises reacting 1-(3-(protected)hydroxybutyl)-4-piperidinyl methylaamine with methyl 3,4-dihydro-2H-[1,3]oxazino[3,2-α]indole-10-carboxylate and subsequently removing the protecting group.

5. A pharmaceutical composition comprising a compound according to claim 1, and a pharmaceutically acceptable carrier.

6. A compound according to claim 1 for use as an active therapeutic substance.

7. The use of a compound according to claim 1 in the manufacture of a medicament for use as a 5-HT₄ receptor antagonist.

8. The use according to claim 7 for use as a 5-HT₄ antagonist in the treatment or prophylaxis of gastrointestinal disorders, cardiovascular disorders and CNS disorders.

9. A method of treatment of irritable bowel syndrome, gastro-oesophagal reflux disease, dyspepsia, atrial arrhythmias and stroke, anxiety and/or migraine in mammals, such as humans, which comprises the administration of an effective amount of the compound of the formula (I) or a pharmaceutically acceptable salt thereof.
